(19) United States
(12) Patent Application Publication
(10) Pub. No.: US 2012/0035677 A1
(43) Pub. Date: Feb. 9, 2012

(54) DEFIBRILLATION SYSTEM AND CARDIAC DEFIBRILLATION METHOD

(75) Inventors: Hiroyuki IMABAYASHI, Tokyo (JP); Naoki NARUMI, Tokyo (JP); Takeshi MORI, Tokyo (JP)

(73) Assignee: OLYMPUS CORPORATION, Tokyo (JP)

(21) Appl. No.: 13/152,460

(22) Filed: Jun. 3, 2011

(30) Foreign Application Priority Data
Aug. 6, 2010 (JP) P2010-177757
Dec. 27, 2010 (JP) P2010-290152

(51) Int. Cl. A61N 1/39

(52) U.S. Cl. 607/7; 607/5

(57) ABSTRACT

Provided is a defibrillation system including an electrode section that is attached to a heart and applies electrical energy to the heart, a defibrillator that generates the electrical energy on the basis of a predetermined defibrillation wave, and a lead that electrically connects the electrode section and the defibrillator. Further, the defibrillation wave includes a first wave generating first energy, a second wave generating second energy higher than the first energy of the first wave behind the first wave, and an application stop period formed between the first wave and the second wave.

### BACKGROUND OF THE INVENTION

1. Field of the Invention

The present invention relates to a defibrillation system to be implanted in a human body, and a cardiac defibrillation method.

This application claims priority to and the benefits of Japanese Patent Application No. 2010-177757 filed on Aug. 6, 2010 and Japanese Patent Application No. 2010-290152 filed on Dec. 27, 2010, the disclosure of which is incorporated herein by reference.

2. Background Art

When ventricular fibrillation of cardiac arrhythmias takes place, there is a high possibility of causing death, because pumping of blood from the heart is promptly stopped, and supply of the blood to the whole body becomes insufficient.

To normalize movement of the heart by removing the ventricular fibrillation, use is made of means (defibrillation) for delivering a shock of high energy to the heart, relieving disordered contractions of individual tissue domains, maintaining order in cardiac muscles to reestablish a systematically spreading activity potential, and restoring synchronous contraction of cardiac tissues.

For example, an apparatus (a defibrillation system) that can be implanted in the heart of a patient who is given defibrillation treatment is disclosed in Japanese Unexamined Patent Application Publication No, 2001-514567. This apparatus includes a plurality of main electrodes, at least one auxiliary electrode, a power supply, and a control circuit.

The plurality of main electrodes send a defibrillation pulse (a main pulse) along a predetermined current pathway in a first portion of the heart. When the plurality of main electrodes send the defibrillation pulse, the current pathway defines a weak electric field area in a second portion of the heart. The auxiliary electrode sends an auxiliary pulse to the weak electric field area. An electrical defibrillation pulse including a monophasic auxiliary pulse is continuously sent via the auxiliary electrode, and then a biphasic fibrillation removal pulse is sent via the main electrodes. The fibrillation removal pulse is sent within 20 milliseconds (msec) after the auxiliary pulse is sent, and a first phase of the fibrillation removal pulse has an opposite polarity to the auxiliary pulse.

The control circuit controls the auxiliary pulse such that the auxiliary pulse does not exceed 40% or 50% of peak current, or 20% or 30% of sending energy (calculated in terms of Joules) of the fibrillation removal pulse.

### SUMMARY OF THE INVENTION

The present invention has been made in view of the above-described circumstances, and an object of the invention is to provide a defibrillation system capable of performing defibrillation more reliably without depending on individual differences of patients, further improving defibrillation effects, and initiating treatment of the heart in its early stage.

According to a first aspect of the present invention, there is provided a defibrillation system including an electrode section that is attached to a heart and applies electrical energy to the heart, a defibrillator that generates the electrical energy on the basis of a predetermined defibrillation wave, and a lead that electrically connects the electrode section and the defibrillator. Further, the defibrillation wave includes a first wave generating first energy, a second wave generating second energy higher than the first energy of the first wave behind the first wave, and an application stop period formed between the first wave and the second wave.

In this case, the defibrillation wave may be configured so that a first wave section in which the first wave and the application stop period are formed as a set is set ahead of the second wave a plurality of times.

Further, in the defibrillation system, the first wave may have an application time ranging from 30 msec to 200 msec, and the first wave section may have a period that ranges from 130 msec to 600 msec and that is a sum of the application time of the first wave and the duration time of the application stop period.

Further, in the defibrillation system, the first wave may have a peak voltage ranging from 10 V to 90 V as absolute value, and the second wave may have a peak voltage ranging from 70 V to 500 V as absolute value.

Further, in the defibrillation system, the plurality of first wave sections may be configured so that a polarity of at least one first wave is different from those of the other first waves.

Further, the second wave may be formed by synthesizing a square wave and a biphasic wave continuously.

Further, the waveform of the square wave may be same as the waveform of the first wave.

Further, the defibrillator may comprise a pulse generating section for generating pulses stimulating a heart, a first capacitor for storing electric power for generating the first wave from the pulse generating section, a second capacitor for storing electric power for generating the second wave having a higher voltage than the first wave from the pulse generating section, a voltage applying section for applying voltage to the first and second capacitors, and a controlling section for controlling the pulse generating section. Further, the controlling section may control the pulse generating section so that the pulse generating section outputs the plurality of first waves at time intervals prior to outputting the second wave, and so that a discharge period of the first capacitor overlaps with a charge period of the second capacitor.

Further, the controlling section may control the pulse generating section to output the plurality of first waves at shorter intervals than a refractory period of the heart.

Further, the voltage applying section for applying voltage to the first and second capacitors may be provided in common.

Further, the first capacitor may be provided in plural numbers, and the controlling section may control the pulse generating section to make the discharge periods of the plurality of first capacitors different from one another.

Further, the controlling section may control the voltage applying section to perform charging for at least part of a stop period between the first waves output a plurality of times.

According to a second aspect of the present invention, there is provided a defibrillation system including a pulse generating section for generating pulses stimulating a heart, a first capacitor for storing electric power for generating a first wave from the pulse generating section, a second capacitor for storing electric power for generating a second wave having a higher voltage than the first wave from the pulse generating section, a voltage applying section for applying voltage to the first and second capacitors, and a controlling section for controlling the pulse generating section. Further, the controlling section controls the pulse generating section so that the pulse generating section outputs the plurality of first waves at time intervals prior to outputting the second wave, and so that a discharge period of the first capacitor overlaps with a charge period of the second capacitor.

In this case, the controlling section may control the pulse generating section to output the plurality of first waves at shorter intervals than a refractory period of the heart.

The refractory period of the heart refers to a period immediately after ventricular excitement, and for which the heart does not react to any stimulus.

Further, the voltage applying section for applying voltage to the first and second capacitors may be provided in common.

Further, the first capacitor may be provided in plural numbers, and the controlling section may control the pulse generating section to make the discharge periods of the plurality of first capacitors different from one another.

Further, the controlling section may control the voltage applying section to perform charging for at least part of a stop period between the first waves output a plurality of times.

Further, the defibrillation system may further include a first output terminal outputting the first wave, and a second output terminal outputting the second wave.

According to a third aspect of the present invention, there is provided a cardiac defibrillation method including a first energy application process of applying first energy to a heart, a second energy application process of applying a second energy higher than the first energy to the heart, and an application stop process, set between the first energy application process and the second energy application process, of stopping application of electrical energy for a predetermined time.

In this case, the first energy application process may have an application time ranging from 30 msec to 200 msec, and the sum of the application time and a time of the application stop process may range from 130 msec to 600 msec.

Further, the first energy application process may have a peak voltage ranging from 10 V to 90 V as absolute value, and the second energy application process may have a peak voltage ranging from 70 V to 500 V as absolute value.

### BRIEF DESCRIPTION OF THE DRAWINGS

FIG. 1A shows a schematic configuration of a defibrillation system according to a first embodiment of the present invention.

FIG. 1B is a cross-sectional view taken along line A-A of FIG. 1A.

FIG. 2 is an enlarged view of a first electrode in an electrode section of the defibrillation system according to the first embodiment of the present invention.

FIG. 3 shows an electrocardiographic waveform in the event of ventricular fibrillation.

FIG. 4 is a schematic view showing generating and meandering of spiral re-entries in the heart.

FIG. 5 shows a defibrillation wave of the defibrillation system according to the first embodiment of the present invention.

FIG. 6 is a schematic view showing a state where spiral re-entries move from a cardiac deep part to a cardiac outer layer part.

FIG. 7 shows an electrocardiographic waveform when the defibrillation system according to the first embodiment of the present invention performs defibrillation.

FIG. 8 shows a schematic configuration of a defibrillation system according to a second embodiment of the present invention.

FIG. 9 shows an electrode section and a lead of the defibrillation system according to the second embodiment of the present invention.

FIG. 10 shows a defibrillation wave of the defibrillation system according to the second embodiment of the present invention.

FIG. 11 shows a defibrillation wave in a first modification of the first and second embodiments of the present invention.

FIG. 12 shows a defibrillation wave in a second modification of the first and second embodiments of the present invention.

FIG. 13 shows a defibrillation wave in a third modification of the first and second embodiments of the present invention.

FIG. 14 shows a defibrillation wave in a fourth modification of the first and second embodiments of the present invention.

FIG. 15 shows an overall configuration of a defibrillation system according to a third embodiment of the present invention.

FIG. 16 is a timing chart showing a relationship between a charge timing and a pulse output timing with respect to capacitors of the defibrillation system according to the third embodiment of the present invention.

FIG. 17 is a timing chart showing operation of switches of the defibrillation system according to the third embodiment of the present invention.

FIG. 18 is a flowchart showing a process performed by a control unit of the defibrillation system according to the third embodiment of the present invention.

FIG. 19 shows an output timing of a first waveform pulse in the defibrillation system according to the third embodiment of the present invention.

FIG. 20 shows an overall configuration of a defibrillation system according to a first modification of the third embodiment of the present invention.

FIG. 21 shows an overall configuration of a defibrillation system according to a second modification of the third embodiment of the present invention.

FIG. 22 shows an overall configuration of a defibrillation system according to a third modification of the third embodiment of the present invention.

FIG. 23 is a timing chart showing a relationship between a charge timing and a pulse output timing with respect to capacitors of the defibrillation system according to the third modification of the third embodiment of the present invention,

### BEST MODE FOR CARRYING OUT THE INVENTION

Recently, by using an optical mapping method, reentries (spiral reentries) caused by spiral waves as excitation wave generated from a heart are regarded as one of the causes that generates ventricular fibrillation. It has been known that such spiral reentries move in the heart while performing wandering motion (meandering), and are further divided serially, so that the entire ventricle leads to a fibrillated state.

Recently, a biphasic defibrillation wave (its waveform in which a polarity of a pulse is reversed in a short time) has been frequently used. However, in a first defibrillation based on electrical energy set for a defibrillator, the ventricular fibrillation might not be completely stopped according to the conditions of a patient, and thus second defibrillation may be performed with higher electrical energy. Successful defibrillation as soon as possible after the ventricular fibrillation occurs is regarded as important for improvement of a quality of life (QOL) of the patient in the future. Above all, when the defibrillation is performed with high electrical energy, heat generated from electrodes has an influence on surrounding biological tissues. For this reason, from the viewpoint of reducing invasion of a human body, it is preferable to perform the defibrillation using minimum necessary electrical energy.

The defibrillation system of the present invention is constructed to be able to ensure a higher probability of successful defibrillation on the basis of the discovery described above. Hereinafter, each embodiment will be described in detail.

### First Embodiment

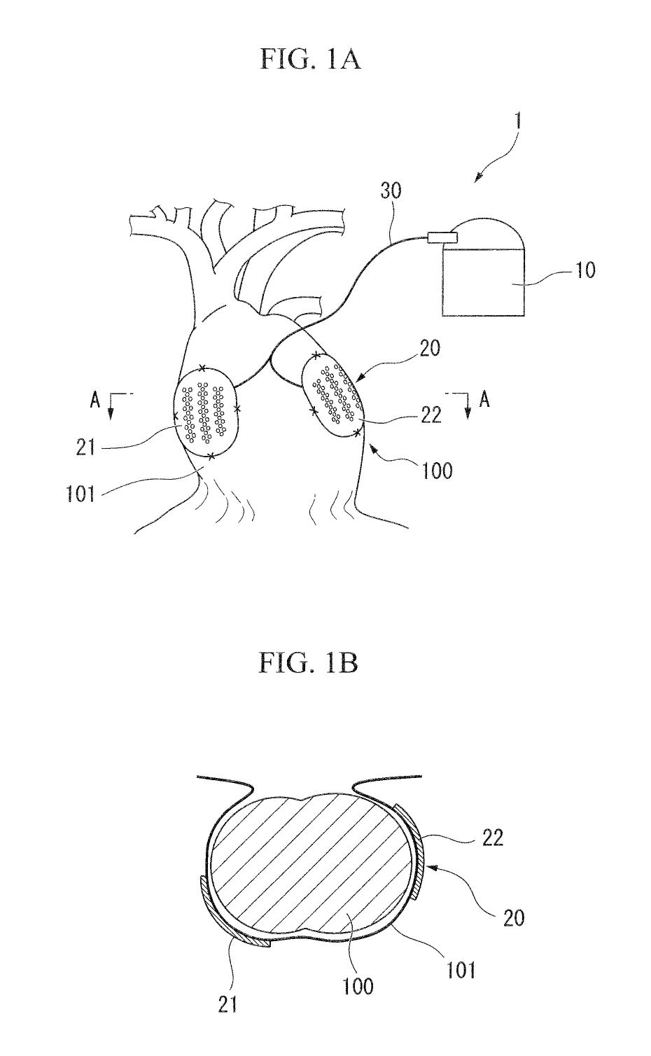

FIG. 1A shows a schematic configuration of a defibrillation system 1 according to a first embodiment of the present invention. FIG. 1B is a cross-sectional view taken along line A-A of FIG. 1A. As shown in FIGS. 1A and 13, the defibrillation system 1 includes a defibrillator 10 that generates electrical energy for defibrillation, an electrode section 20 attached to a heart 100, and a lead 30 that connects the defibrillator 10 and the electrode section 20.

The defibrillator 10 is equipped therein with a variety of components (none of which is shown) such as a battery serving as a power supply, a capacitor storing electrical energy, a detection circuit detecting an electrocardiogram, a determination circuit determining conditions of the heart on the basis of the electrocardiogram, and a defibrillation drive circuit discharging the energy from the capacitor.

The electrical energy is discharged from the defibrillation drive circuit on the basis of a predetermined defibrillation wave, which will be described below in detail.

The electrode section 20 includes a first electrode 21 and a second electrode 22, both of which have the same structure. As shown in FIGS. 1A and 1B, the first electrode 21 is installed on top of a pericardium 101 on the side of a right ventricle, and the second electrode 22 is installed on top of the pericardium 101 on the side of a left ventricle to be opposite to the first electrode 21 with the heart 100 interposed therebetween (see FIG. 1B).

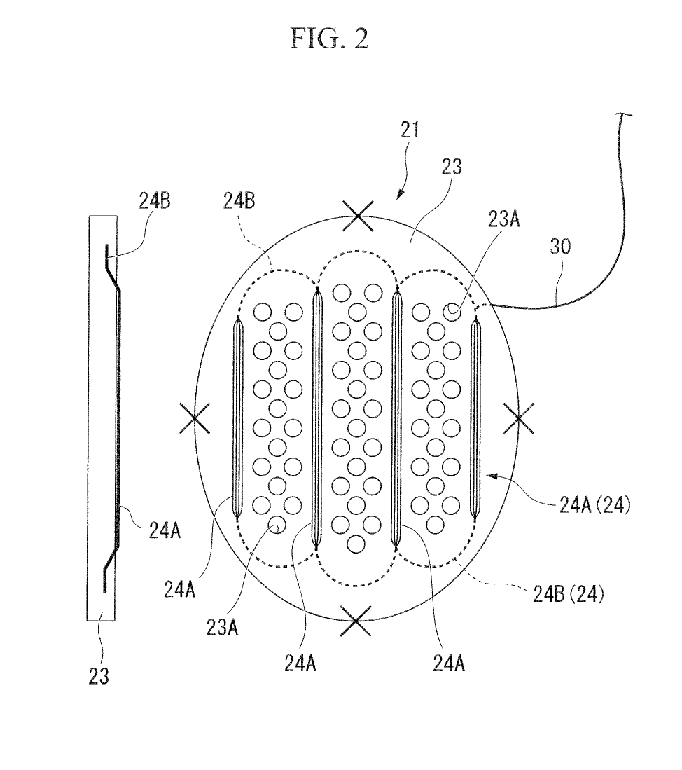

FIG. 2 is an enlarged view of the first electrode 21. The first electrode 21 includes an insulating member 23 of a sheet shape, and an applying lead 24 attached to the insulating member 23.

As a material for forming the insulating member 23, a material having elasticity, flexibility, and insulativity, as well as biocompatibility, is preferable. In this embodiment, silicon is used. A thickness of the insulating member 23 is set to have a maximum value of, for example, about 5 millimeters (mm). In this embodiment, the insulating member 23 is formed in an approximately elliptical shape. However, the present invention is not substantially limited to such a shape.

The applying lead 24 is formed of a strand of a platinum-based material, and preferably a platinum-iridium alloy, and is electrically connected with the lead 30. The applying lead 24 of this embodiment is configured of, for instance, a strand having a diameter of about 0.2 mm so as to be flexible enough to cover an uninterrupted change in shape of the pericardium accompanied with beating of the heart. The applying lead 24 includes four linear parts 24A and curved parts 24B, and is attached to the insulating member 23 to be insulated against internal organs in such a manner that the linear parts 24A are exposed to one surface of the insulating member 23, and that the curved parts 24B are embedded in the insulating member 23. The linear parts 24 are made up of four strands respectively, and are disposed parallel to each other. All the strands constituting the linear parts 24A are connected to the curved parts 24B. Even when one of the strands is disconnected, the electrical energy can be positively supplied to the heart.

In the insulating member 23, a plurality of through-holes 23A are formed in an area between an adjacent two of the four linear parts 24A to increase the flexibility of the insulating member and reduce a contact area with the pericardium. In this embodiment, as shown in FIG. 2, each through-hole has a circular shape. However, the present invention is not limited to such a shape. Further, as long as the first or second electrode 21 or 22 can maintain a constant strength required as an electrode, no restriction is placed on the number of through-holes 23A either.

The second electrode 22 has nearly the same structure as the first electrode except the connected position of the lead 30, and so a description thereof will be omitted. The first and second electrodes 21 and 22 configured as described above are very rich in flexibility as a whole, and have rigidity providing a low possibility of impeding the beating of the heart 100 to cause arrhythmia even when installed on the pericardium 101.

As long as the lead 30 is formed of a material that can maintain electrical insulation to transfer electrical energy from the defibrillator 10 to the electrode section 20, the lead 30 is not substantially limited in its configuration. In the present embodiment, for example, the lead 30 is configured so that an MP35N alloy wire, in the center of which a core wire contains 41% silver, is wound in a polyurethane tube having an outer diameter of about 2 mm in a coil shape. In this configuration, since the MP35N alloy wire is formed in the coil shape, the lead 30 has strong tensile strength and bending strength.

As shown in FIG. 2, the lead 30 is connected to at least one of the curved parts 24B of the applying lead 24 from a rear surface of the insulating member 23 which is opposite to a surface to which the linear parts 24A of the insulating member 23 are exposed. The lead 20 and the applying lead 24 are firmly fixed by welding or mechanical caulking. This joint is covered with the insulating member 23, and thus is not exposed.

A description will be made of how the defibrillation system 1 configured as described above operates at the time of use.

Before the defibrillation system 1 is used, the electrode section 20 is installed on the pericardium 101 on the surface of the heart 100 of a patient. The electrode section 20 is installed on the pericardium 101 by causing a suture to pass through four places to be sutured within the insulating member 23 indicated in FIG. 2 by a mark X. The suture is performed by causing a needle such as a curved needle and a suture to pass through in a thickness direction of the insulating member 23 so as to pierce only the pericardium 101 but not the cardiac muscle of the heart. Thereby, the electrode section 20 is fixed only to the pericardium 101. Accordingly, the heart 100 can freely move inside the pericardium 101 without being influenced by the electrode section 20, and a risk of generating the arrhythmia due to impedance of the movement of the heart can be inhibited.

The electrode section 20 may be installed under thoracotomy, or under thoracoscopy using, for instance, a trocar. However, from the viewpoint of inhibiting invasion of a patient, the electrode section 20 is preferably installed under thoracoscopy. Since the first electrode 21 and the second electrode 22 have excellent flexibility, when installed under thoracoscopy, the electrodes may be installed by merely rolling up the electrodes to be able to pass through the trocar, delivering them into a pleural cavity, and rolling them out in the pleural cavity in a planar shape.

The lead 30 drawn out of the human body and the defibrillator 10 may be held outside the human body or may be embedded under the skin.

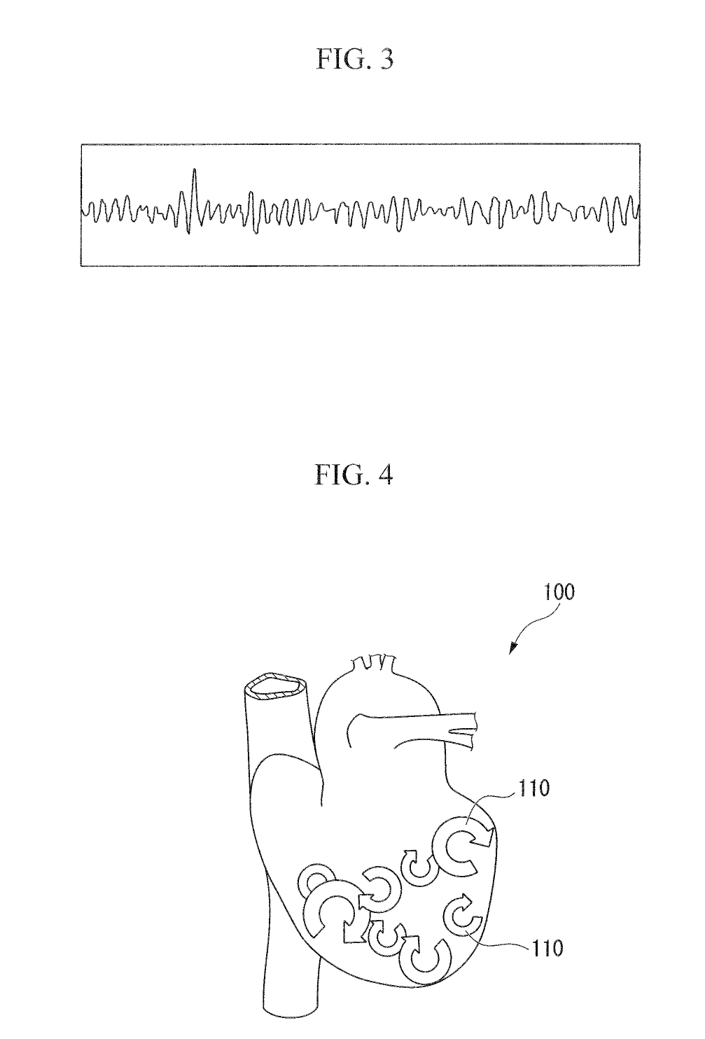

The defibrillation system 1 attached to the patient always monitors an electrocardiogram of the patient using the detection circuit installed in the defibrillator 10. When ventricular fibrillation is generated during monitoring, the electrocardiogram of the ventricular fibrillation is obtained as shown in FIG. 3, and thus generation of the ventricular fibrillation is detected.

When the ventricular fibrillation is generated, the heart beats violently over 200 times per minute. The beats are irregular, and a magnitude or period of a wave of the electrocardiogram is inconsistent. This is because a plurality of spiral reentries 110 are meandering as shown in FIG. 4, and one cause is considered to be that electrical stimuli are randomly transferred from the surface or interior of the heart 100, and thus each region is contracted at random.

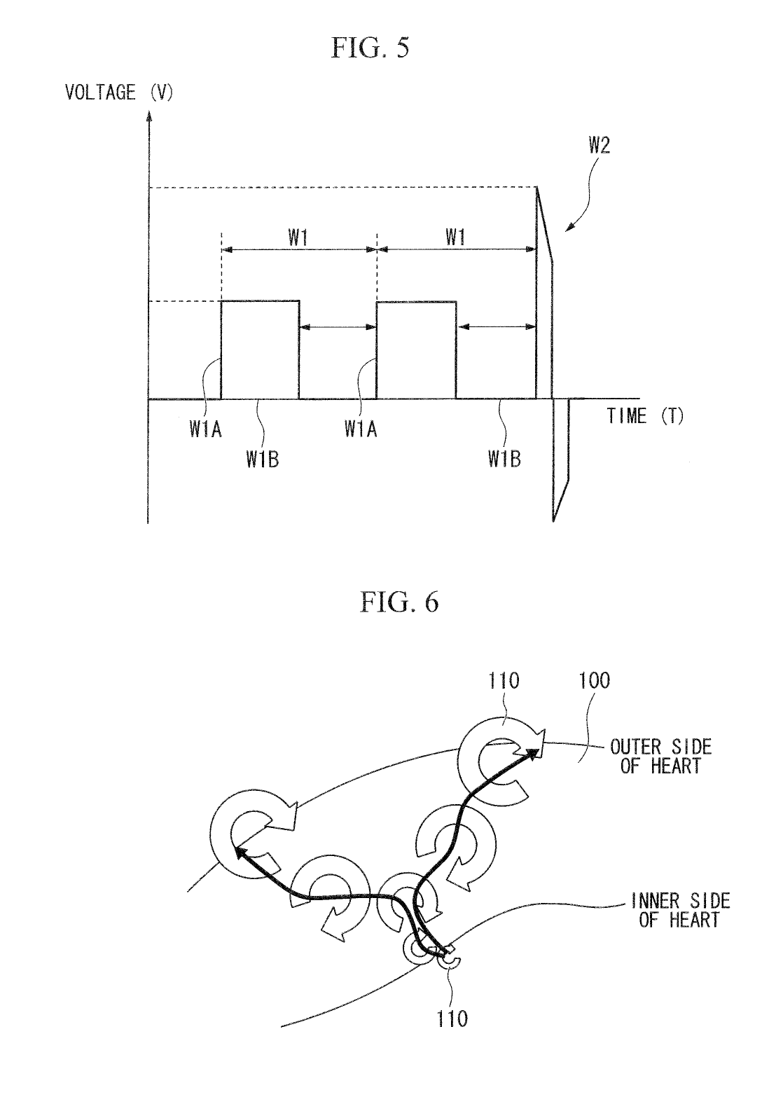

In the defibrillation system 1 of the present embodiment, when the generation of the ventricular fibrillation is detected, the defibrillation drive circuit of the defibrillator 10 generates electrical energy on the basis of a defibrillation wave as shown in FIG. 5.

The generated electrical energy is transferred to the electrode section 20 through the lead 30, and thus is applied between the first electrode 21 and the second electrode 22.

The defibrillation wave generated from the defibrillation drive circuit includes a first wave section W1 that uniformizes a period of a spiral reentry (hereinafter, acronymized as “SR”) in the heart, and a second wave W2 that removes ventricular fibrillation following the first wave section W1. In the present embodiment, immediately after the first wave section W1 repetitively acts on the heart twice, electrical energy (second energy) generated on the basis of the second wave W2 is applied to the heart. Thereby, a series of defibrillation processes are performed.

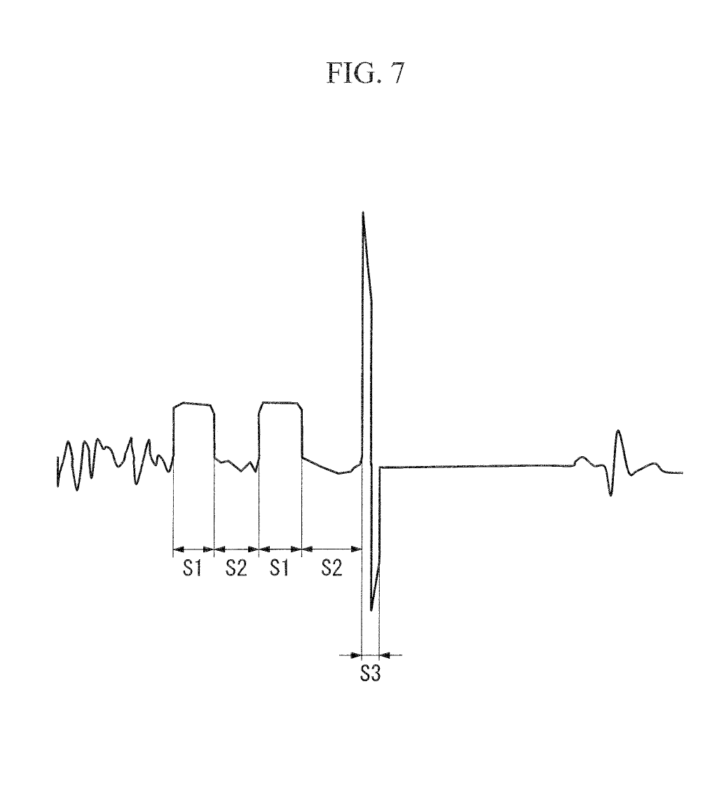

The first wave section W1 includes a square wave (a first wave) W1A generating electrical energy (first energy) having a predetermined magnitude, and an application stop period W1B following the square wave W1A. When the square wave W1A is applied to the heart, the SRs generated in an outer layer and a relatively shallow region of the heart 100 (hereinafter, generally referred to as an “outer layer part”) are attracted to and captured by a potential of the square wave W1A, and thus the SRs stop meandering (a process of applying the first energy: S1 in the electrocardiogram waveform shown in FIG. 7).

When the application of the square wave W1A is terminated, the application stop period W1B is initiated, and the capture of the SRs is released. However, as the SRs are captured for a predetermined time, re-excitation is not input, and thus the SRs are dissipated. Thereby, most of the SRs generated in the outer layer part of the heart 100 are dissipated (a process of stopping application of energy: S2 in the electrocardiogram waveform shown in FIG. 7). Furthermore, as shown in FIG. 6, most of the SRs 110 generated from a deep part of the cardiac muscle which is adjacent to the ventricle or a cardiac septum (hereinafter, generally referred to as a “cardiac deep part”) are propagated from the cardiac deep part to reach the outer layer part during application of the square wave W1A, and are also captured and dissipated by the square wave W1A. That is, when the square wave W1A is applied to the heart, the SRs that are low in synchronism with an application period of the square wave W1A are considerably dissipated.

The SRs of the outer layer part are dissipated by the square wave W1A generating a lower electrical energy than the second wave W2, but the SRs that are high in synchronism with the application period of mainly the square wave W1A at the deep part of the cardiac muscle which is adjacent to the ventricle or the cardiac septum are left. These SRs are propagated up to the outer layer part for the application stop period W1B, as shown in FIG. 6, but a disordered state thereof is improved compared to before the square wave W1A is applied. The SRs are arranged with a constant period to some extent regardless of an individual difference of the patients. In the present embodiment, the first wave section W1 is applied twice, thereby further reducing the total amount of SRs of the heart, and further increasing the degree to which the period is arranged. Then, the second wave W2 is applied.

The second wave W2 is a biphasic wave as disclosed in Japanese Unexamined Patent Application Publication No. 2001-514567. In the present embodiment, the second wave W2 is applied immediately after the application stop period W1B of the second first wave section W1 is terminated (a process of applying second energy: S3 in the electrocardiogram waveform shown in FIG. 7). As such, the electrical energy is applied to the heart such that it is synchronized to the ventricular fibrillation period arranged by the first wave section W1, and thus the defibrillation is performed. In other words, the electrical energy of the second wave W2 can be applied at a timing adjacent to when the entire heart is electrically excited to the utmost by the SR whose period is arranged to some extent. Thereby, it is possible to easily allay the electrical excitement of the heart, and the probability of success of the defibrillation is improved. In the case of an electrocardiographic waveform shown in FIG. 7, after the process of applying second energy, the beats of the heart are normalized.

In the present embodiment, the second wave W2 may have, for example, a positive-side peak voltage of 160 volts (V), an energized time of 6 msec, a negative-side peak voltage of 100 V, and an energized time of 6 msec. In the ordinary defibrillators, it is difficult or next to impossible to remove the ventricular fibrillation by using a biphasic wave having this magnitude. In the defibrillation system 1 of the present embodiment, a defibrillation wave combining the first wave section W1 and the second wave W2 is used, so that, compared to a conventional defibrillator, a peak voltage value of the biphasic wave in the second wave W2 is controlled to be low, thereby making it possible to perform the defibrillation.

For example, when a threshold of a defibrillation energy caused by a biphasic wave as disclosed in Japanese Unexamined Patent Application Publication No. 2001-514567 is about 3 Joules (J), the electrical energy of the second wave W2 of the present embodiment can be reduced by about ⅓ to about ½ (about 1 J to about 1.5 J) of the threshold. Accordingly, it is possible to remarkably reduce an impulse to the patient generated along with the defibrillation, and inhibit the invasion to improve QOL of the patient on whom the defibrillation system 1 is being mounted.

Further, in the defibrillation system of the present invention, the defibrillator is preferably configured to be able to set the peak voltage of the second wave within a range from 70 V to 500 V as absolute value. If the peak voltage of the second wave can be set within this range, it is possible to suitably cope with the case where the electrode section is installed on the pericardium as in the present embodiment as well as the case where the electrode section is installed in the heart transvenously.

In the present embodiment, a voltage value and a length (time) of the first wave section W1 are also important, and are preferably set to a predetermined range.

When the voltage value of the square wave W1A is too low, it is difficult to capture the SR in the state where the meandering of the SR is stopped, and the area of a region where the SR is captured in the heart also becomes small. Meanwhile, when the voltage value is too high, it is difficult to arrange the period of the SR because even the SR of the deep part is captured.

Taking this into consideration, the voltage value of the square wave W1A preferably ranges from 10 V to 90 V, and more preferably from 40 V to 60 V.

When an application time of the square wave W1A is too short, it is possible to capture the SR only for a moment, it is difficult to control re-excitement to be low, and thus it is difficult to dissipate the SR. In contrast, when too long, a time for which the heart is stopped becomes long, and a burden of the patient increases. The application time of the square wave W1A preferably ranges from 30 msec to 200 msec, and more preferably from 50 msec to 100 msec.

When the application stop period W1B is too short, the SR generated from the cardiac deep part or the cardiac septum does not reach the outer layer part, and the effects of the first or second wave applied subsequently become weak. In contrast, when the application stop period W1B is too long, the SR is further increased with expanding after it reaches the outer layer part, and thus the state becomes similar to before the square wave W1A is applied. Accordingly, the length of the application stop period W1B is preferably set to be approximately equal to a time for which the SR generated from the cardiac deep part or the cardiac septum is required to reach the outer layer part. In order for the SR generated from the cardiac deep part to arrive up to the outer layer part through the process shown in FIG. 6, a time ranging from about 100 msec to about 400 msec is required even if an individual difference is present. Accordingly, the length of the application stop period W1B preferably ranges from 100 msec to 400 msec, and more preferably from 100 msec to 200 msec.

From the foregoing, the period T of the first wave section W1 that is the total sum of the square wave W1A and the application stop period W1B preferably ranges from 130 msec to 600 msec, and more preferably from 150 msec to 300 msec.

As described above, according to the defibrillation system 1 of the present embodiment, since the defibrillation wave generated from the defibrillation drive circuit of the defibrillator 10 includes the first wave section W1 and the second wave W2, it is possible to more positively perform the defibrillation regardless of individual differences of the patients. Further, it is possible to perform the defibrillation with a less energy than a conventional defibrillation system. For this reason, it is possible to reduce the invasion of the patient, and to further prolong a period of time (durability) from a time when the system is implanted to a time when it is necessary to exchange a battery. According to the defibrillation system I of the present embodiment, it is unnecessary to install auxiliary electrodes in the heart as disclosed in Japanese Unexamined Patent Application Publication No. 2001-514567, and thus it is possible to reduce a burden on the heart,

In the present embodiment, the example has been described so that the first wave section W1 is applied twice before the second wave W2 is applied. However, depending on the degree of a heavy injury of the ventricular fibrillation or conditions of the patient, the first wave section W1 may be applied once before the second wave W2 is applied, or the first wave section W1 may be applied three times or more. As the number of times the first wave section W1 is applied to is increased, the total number of the SRs decreases, and the period of the SR is gradually arranged equally. As such, as long as the defibrillation does not depart from its original purpose of rescuing the patient's life, it is preferable that the number of times the first wave section W1 of the defibrillation wave is applied to is as many as possible.

### Second Embodiment

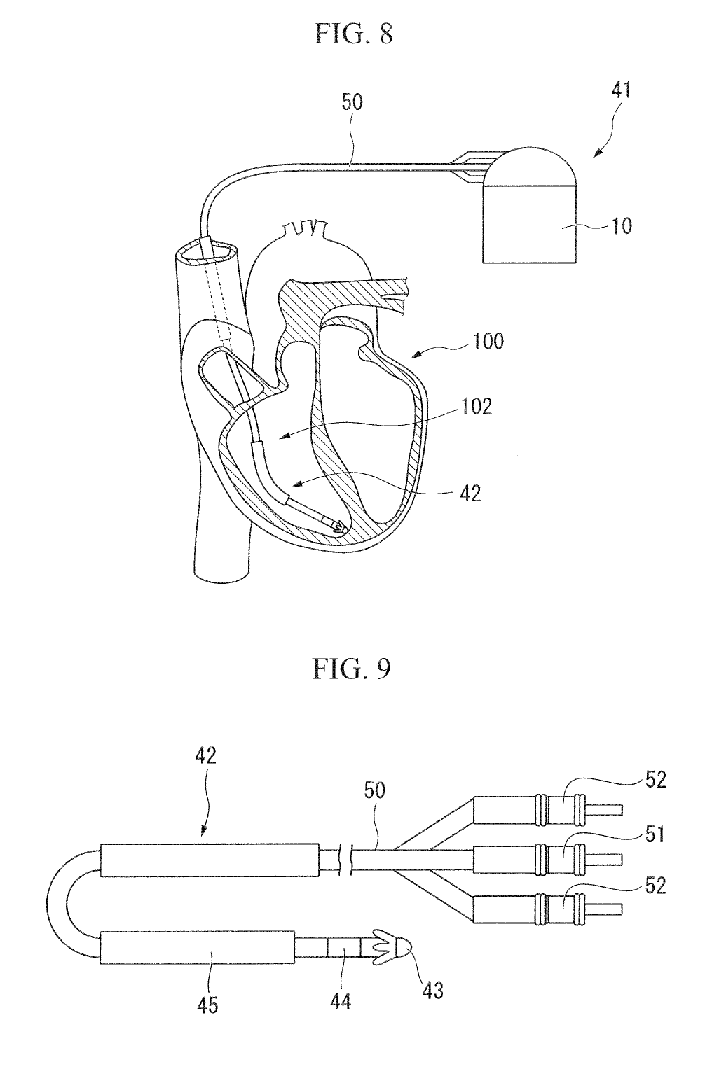

Next, a second embodiment of the present invention will be described with reference to FIGS. 8 through 11. Differences between a defibrillation system 41 of the present embodiment and the aforementioned defibrillation system 1 are a shape of the electrode section, a shape of the lead, and the waveform of the defibrillation wave. Further, the components common to the foregoing first embodiment are represented by the same numerals and symbols, and so a repetitive description thereof will be omitted.

FIG. 8 shows an electrode section 42 and a lead 50 of the defibrillation system 41. The electrode section 42 and the lead 50 are generally designated as a “right ventricle (RV) lead (or a transvenous lead).” As shown in FIG. 9, a chip electrode 43 and a ring electrode 44, both of which are used to detect an electrocardiogram or perform cardiac pacing, are installed near a leading end of the electrode section 42. The electrode section 42 is provided with an RV defibrillation (RV-def) electrode 45 at an intermediate portion thereof to which a defibrillation wave is applied. Each electrode of the electrode section 42 and the lead 50 are electrically connected by intra-electrode leads (not shown). Further, the lead 50 is connected to the defibrillator 10 by an IS1 connector 51 and a pair of DF1 connectors 52, all of which are provided at a proximal end of the lead 50.

Unlike the electrode section 20 of the first embodiment, the electrode section 42 is delivered up to the heart 100 via a blood vessel using, for instance, a catheter. The RV-def electrode 45 is installed to be generally located in a right ventricle 102 with the electrode section 42 implanted.

In the defibrillation system 41, a defibrillation wave generated from a defibrillation drive circuit is applied between the defibrillator 10 and the RV-def electrode 45.

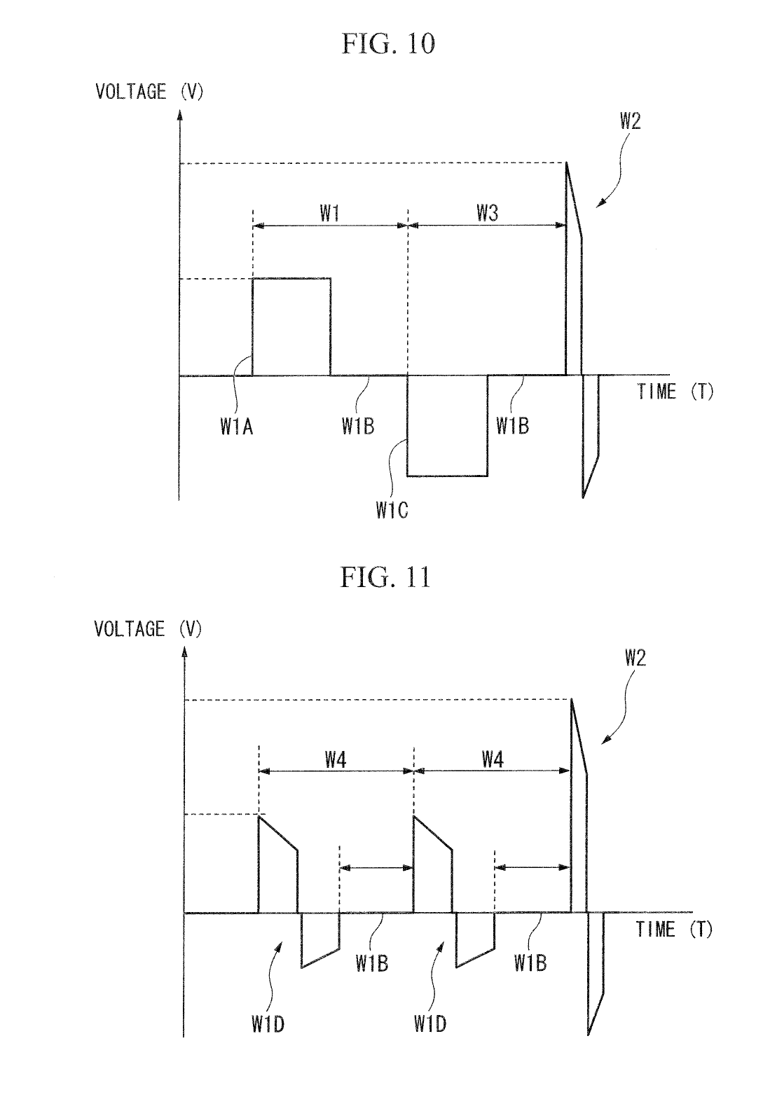

FIG. 10 shows the defibrillation wave of the defibrillation system 41. In the defibrillation system 41 as well, a first wave section is applied twice. However, as shown in FIG. 10, in a second first wave section W3, a square wave W1C having a polarity different from that of a square wave W1A is applied. The square wave W1C has a voltage value of negative 40 V, and its absolute value is set to be equal to that of the square wave W1A.

In the present embodiment, in the first wave section W3 applied for the second time, a square wave W3A, which has a negative polarity inverting a polarity of first energy and has an absolute value of a voltage substantially equal to that of the square wave W1A, is applied. Thereby, the positive-polarity electrification hardly takes place at the heart 100. Even when the applied electrical energy has a negative polarity, SRs generated in an outer layer part of the heart 100 can be captured while stopping meandering, like the electrical energy of a positive polarity. After the square wave W3A is applied, the captured SRs are also dissipated, and thus the total number thereof is reduced.

In the defibrillation system 41 of the present embodiment as well, like the defibrillation system 1 of the first embodiment, it is possible to perform the defibrillation more positively by means of reduction in the total number of SRs and synchronization between the ventricular fibrillation period and the applied timing of the second wave.

Further, since the first wave section W3 including the negative-polarity square wave W1C is applied subsequent to the first wave section WI, it is possible to apply an exact polarity voltage to the heart 100 in the event of the defibrillation based on the second wave W2. As a result, it is possible to cancel a bias voltage remaining at the heart to prevent the voltage shift of a biphasic wave.

Further, in the present embodiment, the example has been described so that the first wave section W1 is set ahead of the first wave section W3. Needless to say, it is possible to obtain similar effects even if the sequence is reversed.

While the first and second embodiments of the present invention have been described, the technical scope of the present invention is not limited to these embodiments. Thus, various modifications can be made in the scope without departing from a gist of the present invention.

First, the defibrillation wave generated from the defibrillation drive circuit of the defibrillator can be diversified in addition to those shown in the aforementioned embodiments.

### First Modification of First and Second Embodiments

FIG. 11 shows a defibrillation wave according to a first modification of the first and second embodiments. In this modification, as the first wave of a first wave section W4, a biphasic wave W1D is used in place of the square wave W1A or W3A. However, the biphasic wave W1D has a voltage value and a total application time that are similar to those of the square wave W1A, a lower voltage value than the second wave W2, and a longer application time than the second wave W2.

In this defibrillation wave, as well as in each of the aforementioned embodiments, it is possible to perform the defibrillation more positively compared to a conventional defibrillation system. Further, the fact that the positive-polarity electrification is suppressed at the heart 100 is similar to the second embodiment.

Further, since the first wave is the same biphasic wave as the second wave, it is possible to further simplify the configuration of the defibrillation drive circuit of the defibrillator 10, and to reduce the cost of production.

### Second Modification of First and Second Embodiments

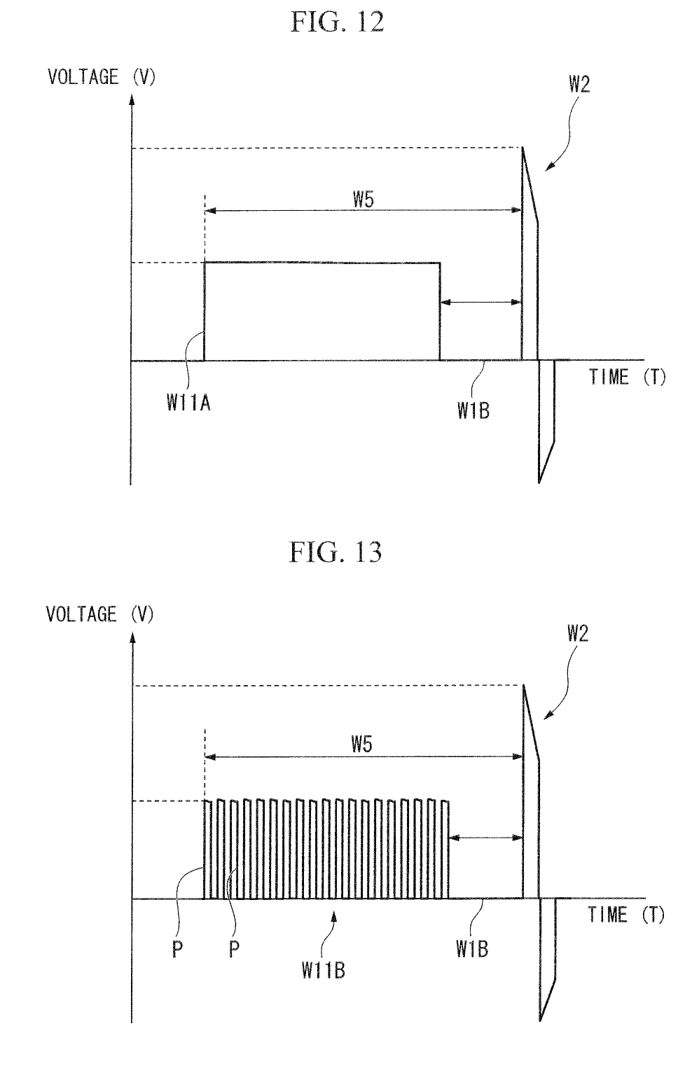

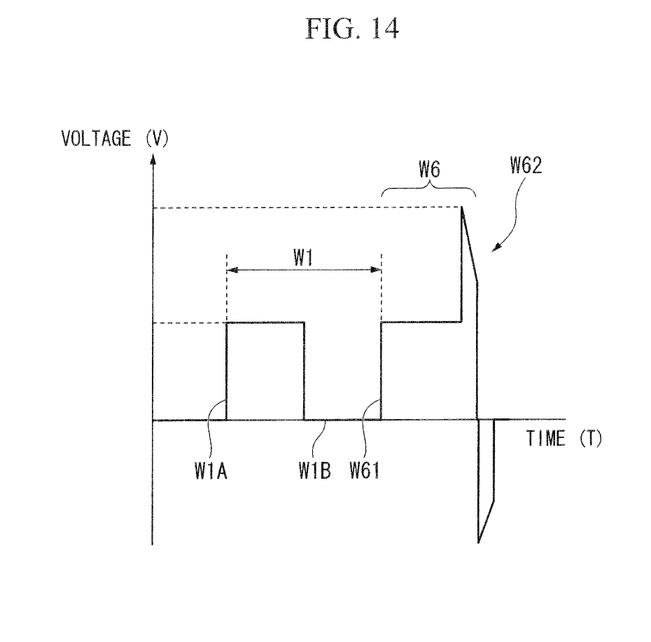

FIG. 12 shows a defibrillation wave according to a second modification of the first and second embodiments. This modification shows an example where a first wave section W5 having a single first wave and a single application stop period in front of a second wave W2 is set only once. A square wave W11A applied as a first wave has a voltage value of positive 40 V that is similar to the square wave W1A, and an application time set to 400 msec that is longer than that of the square wave W1A. The application time of the square wave W11A is preferably set to be slightly longer than the preferable range of the application time when the aforementioned first wave section is applied twice. When the defibrillation wave is set in this way, the effects are slightly less than those of each of the aforementioned embodiments, but it is possible to reduce the total number of SRs prior to applying the second wave, and to perform the defibrillation more positively with less energy than a conventional defibrillation system.

### Third Modification of First and Second Embodiments

Further, as in a third modification shown in FIG. 13, the first wave may be made up of a plurality of pulses P whose application time is sufficiently short. In this modification, 19 pulses are generated in such a manner that the application time of each pulse P is set to 10 msec, and that an interval between the pulses P is set to 10 msec. Thereby, a first wave W11B is formed, which can be regarded as a single square wave whose total application time amounts to 370 msec. According to the present modification, it is possible to reduce consumption power of the first wave W11B to about ½ of the first wave W11A, and to improve a service life of the battery of the defibrillator, while obtaining effects similar to the modification shown in FIG. 12. When the first wave is made up of a plurality of pulses, the interval between the pulses may be set to merely be sufficiently faster than a meandering speed of the SR. The application time of each pulse and the interval between the pulses are not limited to the foregoing. For example, both the application time of each pulse and the interval between the pulses may be set to 1 msec. In the case of a pulse train of several msec, it is possible to obtain approximately similar effects. This first wave may be used to set the first wave section a plurality of times.

### Fourth Modification of First and Second Embodiments

FIG, 14 shows a defibrillation wave according to a fourth modification of the first and second embodiments. This modification shows an example where a second wave W6 is applied in place of the second wave W2 and the first wave W1 is applied once before the second wave W6.

The second wave W6 is formed by synthesizing a square wave W61 and a biphasic wave W62 continuously. The square wave W61 has a voltage value of positive 40 V and a total application time that are similar to those of the square wave W1A. That is, the waveform of the square wave W61 is same as the waveform of the aquare wave W1A. The waveform of the biphasic wave W62 is same as the waveform of the second wave W2. In this modification, the period of the first wave section W1 preferably ranges from 130 msec to 600 msec, and more preferably from 150 msec to 300 msec.

In this modification, because the biphasic wave W62 is continuously applied subsequent to the square wave W61, the biphasic wave W62 can be applied such that it is synchronized to the ventricular fibrillation period arranged by applying the square wave W1A and the square wave W61 periodically. Further, because the biphasic wave W62 is continuously applied subsequent to the square wave W61, the biphasic wave W62 can be applied with the total number of the SRs decreased by applying the square wave W61. Therefore, it is possible to more positively perform the defibrillation with a less energy than a conventional defibrillation system.

Additionally, the square wave W61 may have a voltage value and a total application time that are different from those of the square wave W1A. The first wave section W1 may be applied plurally before the second wave W6 is applied

Further, an application stop time differently from the application stop period may be set between the first wave section applied immediately before the second wave and the second wave. In this case, since the sum of the first wave section and the application stop time can be substantially regarded as the period of the first wave section, the period regarded in this way is included in the present invention if it falls within the aforementioned preferable range, and it is possible to obtain similar effects. This is also applied to the case where the first wave section is made up of a preceding application stop period and a first wave subsequent to it, and an application stop time differently from the first wave section is set between the first wave applied immediately before the second wave and the second wave.

Further, a specific aspect of the configuration described in each of the aforementioned embodiments makes it possible to change a combination of components, or variously modify or eliminate each component, in the scope without departing from the gist of the present invention.

Further, the present invention includes technical ideas described in the following appended claims.

### (Appended Claim 1)

A cardiac defibrillation method comprising:

a first energy application process of applying first energy to a heart;

a second energy application process of applying a second energy higher than the first energy to the heart; and

an application stop process, set between the first energy application process and the second energy application process, of stopping application of electrical energy for a predetermined time.

### (Appended Claim 2)

The cardiac defibrillation method according to the appended claim 1, wherein the first energy application process has an application time ranging from 30 msec to 200 msec, and the sum of the application time and a time of the application stop process ranges from 130 msec to 600 msec.

### (Appended Claim 3)

The cardiac defibrillation method according to the appended claim 1, wherein the first energy application process has a peak voltage ranging from 10 V to 90 V as an absolute value, and the second energy application process has a peak voltage ranging from 70 V to 500 V as an absolute value.

### Third Embodiment

A defibrillation system 2 according to a third embodiment of the present invention will be described below with reference to the figures.

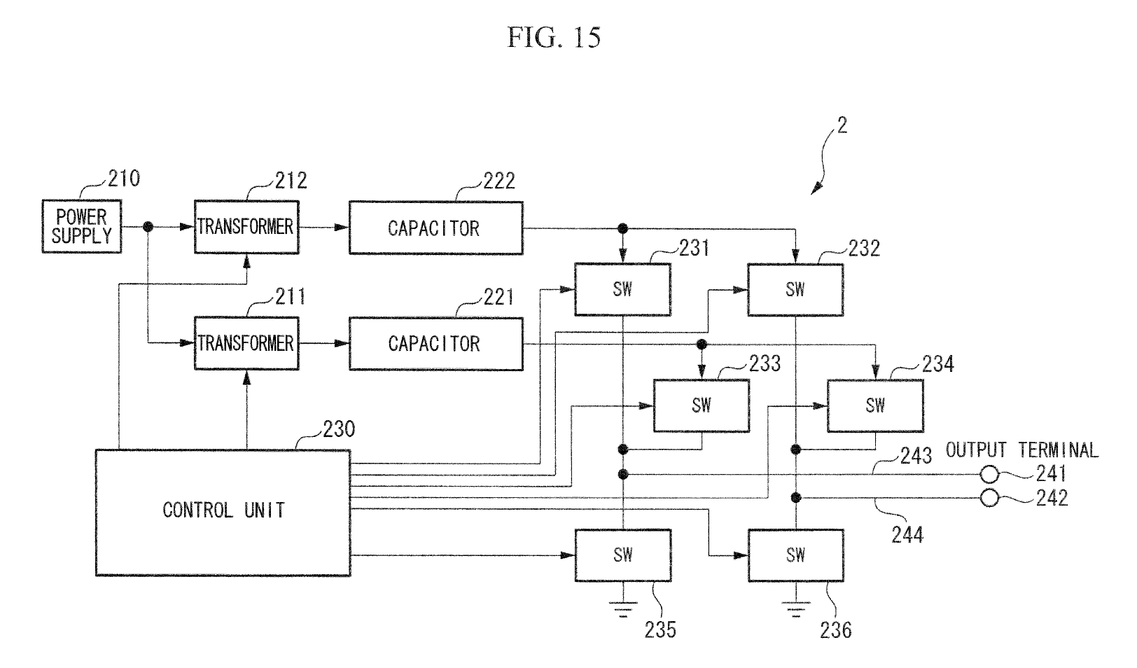

As shown in FIG. 15, the defibrillation system 2 of the present embodiment includes a power supply (a voltage applying section) 210 that applies a voltage, transformers 211 and 212 that convert the voltage applied by the power supply 210, a capacitor (a first capacitor) 221 electrically charged by the application of the voltage from the transformer 211, a capacitor (a second capacitor) 222 electrically charged by the application of the voltage from the transformer 212, output terminals 241 and 242 connected to the outside (electrodes), switches (a pulse generating section) 231 to 236 installed on a circuit between the capacitors 221 and 222 and the output terminals 241 and 242, and a control unit (a controlling section) 230 that controls these components.

One end of a lead (not shown) is connected to the output terminals 241 and 242. The other end of the lead is connected to electrodes (not shown) disposed on the heart. Therefore, a pulse generated from the defibrillation system 2 is transferred to the electrodes disposed on the heart, the heart is stimulated by this pulse, and thus fibrillation of the heart is removed.

The capacitor 221 is connected to the transformer 211, and electric power for generating a first waveform pulse (a first wave) of a low voltage is charged by the voltage applied by the transformer 211. Further, the electric power charged to the capacitor 221 is discharged by turning ON/OFF the switches (hereinafter, acronymized as “SWs”) 231 to 236 as will be described below, and thus the first waveform pulse of the low voltage is output from the output terminals 241 and 242.

The capacitor 222 is connected to the transformer 212, and electric power for generating a second waveform pulse (a second wave) of a high voltage is charged by the voltage applied by the transformer 212. Further, the electric power charged to the capacitor 222 is discharged by turning ON/OFF the SWs 231 to 236 as will be described below, and thus the second waveform pulse of the high voltage is output from the output terminals 241 and 242.

Further, capacity of the capacitor 221 may be set to be greater than that of the capacitor 222, and a voltage drop may be made small by outputting a plurality of pulses by using the discharge of the capacitor 221.

The SWs 231 to 236 are switches disposed on the circuit between the capacitors 221 and 222 and the output terminals 241 and 242, and are turned ON/OFF, thereby converting ON/OFF of each circuit to output the pulses stimulating the heart from the output terminals 241 and 242.

The SWs 231 and 232 are connected to the capacitor 222, and are connected to the SWs 235 and 236 respectively.

The SWs 233 and 234 are connected to the capacitor 221, and are connected to the SWs 235 and 236 respectively.

The SWs 235 and 236 are connected to the ground.

A wiring 243 is connected between the SW 231 and the SW 235 and between the SW 233 and the SW 235, and another end of the wiring 243 is connected to the output terminal 241.

A wiring 244 is connected between the SW 232 and the SW 236 and between the SW 234 and the SW 236, and another end of the wiring 244 is connected to the output terminal 242.

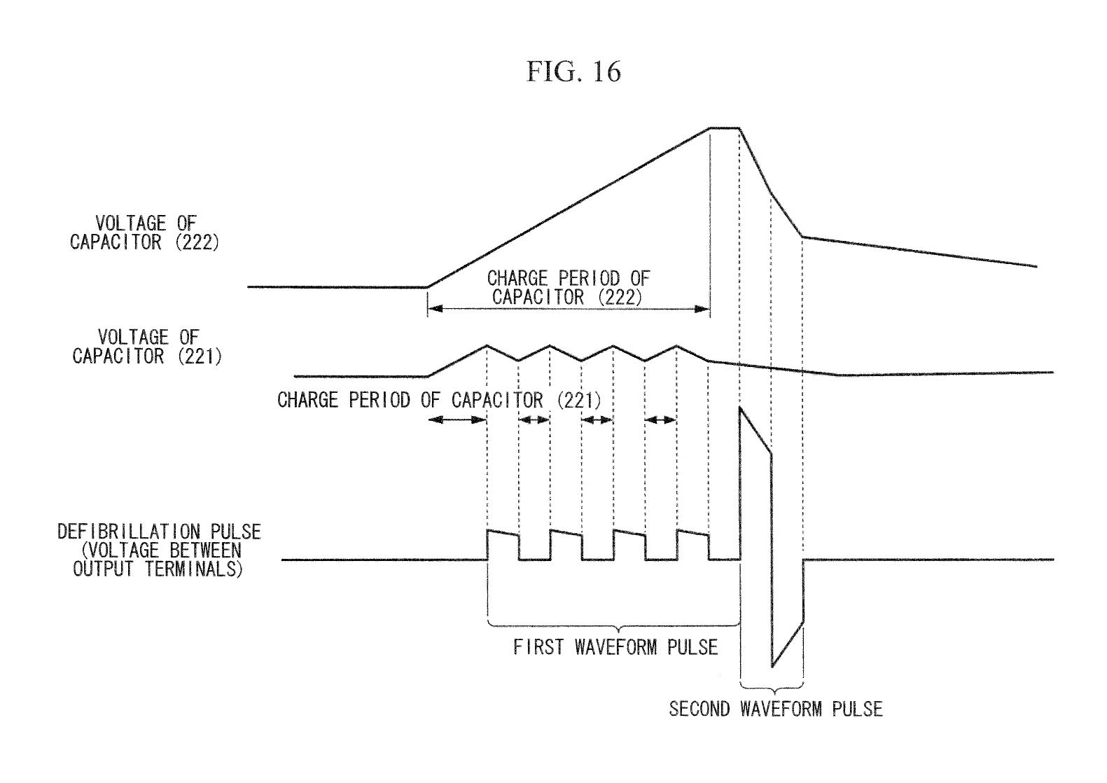

The control unit 230 controls ON/OFF operation of SWs 231 to 236, and controls charging to the capacitors 221 and 222 by means of the transformers 211 and 212. In detail, as shown in FIG. 16, the control unit 230 controls the capacitor 221 to output the plurality of first waveform pulses at time intervals, prior to outputting the second waveform pulse from the capacitor 222. Further, the control unit 230 controls the SWs 231 to 236 and the transformers 211 and 212 such that a discharge period of the capacitor 221 overlaps with a charge period of the capacitor 222.

That is, as shown in FIG. 16, the control unit 230 controls the capacitor 221 to output the first waveform pulse to the output terminals 241 and 242 during the charge period of the capacitor 222, thereby initiating defibrillation treatment. Here, the voltage (a charge voltage) applied to the capacitor 221 ranges, for instance, from 10 V to 90 V, and the voltage (a charge voltage) applied to the capacitor 222 ranges, for instance, from 70 V to 500 V. A magnitude of each charge voltage is relevant to an amplitude of the pulse output. The capacitor 221 is charged for a period when the output of the first waveform pulse is stopped, so that a quantity of discharged charges is charged. In FIG. 16, the charging of the capacitor 221 is performed for all periods when the output of the first waveform pulse is stopped. However, the charging may be performed for a first charge period and a part of stop period between the first waveform pulses, to charge a desired application voltage to the capacitor 221. In this case, it is possible to perform efficient charging for the stop period of the pulses.

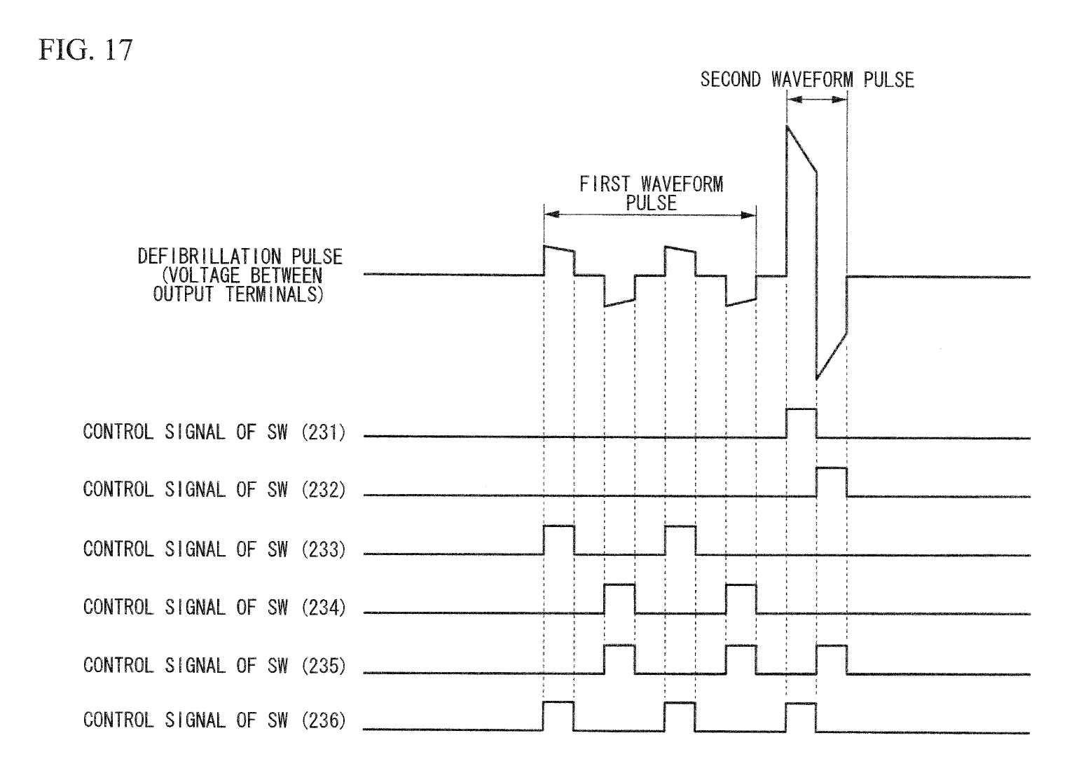

Detailed control of the SWs 231 to 236 on generating the defibrillation pulse will be described with reference to FIG. 17.

FIG. 17 is a timing chart showing detailed ON/OFF operation of the SWs 231 to 236.

As shown in FIG. 17, the first waveform pulse is generated from the capacitor 221 under the control of the SWs 233 to 236. In detail, the SW 233 and the SW 236 are turned ON to generate a positive pulse (a positive-side pulse), and the SW 234 and the SW 235 are turned ON to generate a negative pulse (a negative-side pulse).

Further, the second waveform pulse is generated from the capacitor 222 under the control of the SWs 231, 232, 235 and 236. In detail, the SW 231 and the SW 236 are turned ON to generate a positive pulse (a positive-side pulse), and the SW 232 and the SW 235 are turned ON to generate a negative pulse (a negative-side pulse).

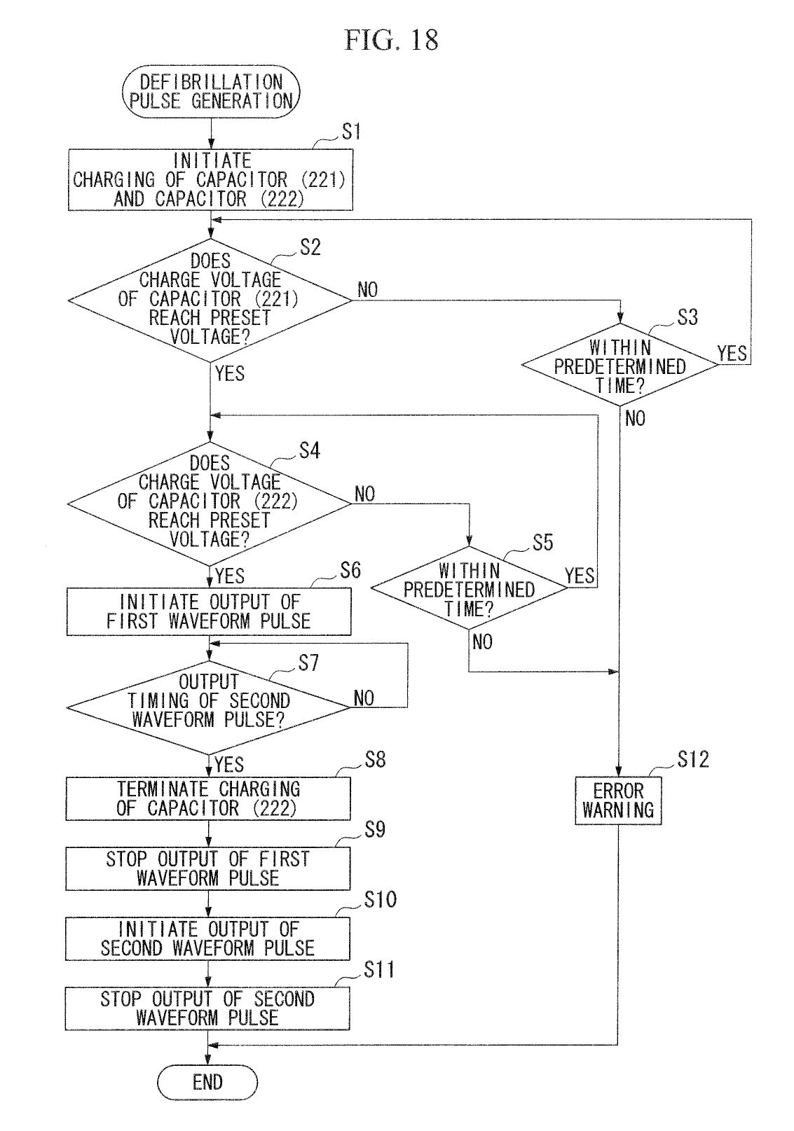

Operation of the defibrillation system 2 of the present embodiment which performs the aforementioned control will be described using a flowchart shown in FIG. 18.

First, when ventricular fibrillation is detected, a defibrillation pulse generating process is initiated, and the charging of the capacitors 221 and 222 is initiated (step S1).

Next, it is determined whether or not the voltage of the capacitor 221 reaches a preset voltage (step S2).

In step S2, when the voltage of the capacitor 221 reaches the preset voltage, it is determined whether or not the voltage of the capacitor 222 reaches a preset voltage (step S4).

Meanwhile, in step S2, when the voltage of the capacitor 221 does not reach the preset voltage within a predetermined time (step S3), it is determined that an abnormality has occurred, an error warning is given (step S12), and the defibrillation pulse generating process is terminated.

In step S4, when the voltage of the capacitor 222 reaches the preset voltage, a first waveform pulse is output (step S6).

Meanwhile, in step S4, when the voltage of the capacitor 222 does not reach the preset voltage within a predetermined time (step S5), it is determined that an abnormality has occurred, an error warning is given (step S12), and the defibrillation pulse generating process is terminated.

Next, after the first waveform pulse is output, it is determined whether or not the output timing of a second waveform pulse has arrived (step S7).

In step S7, when the output timing of the second waveform pulse has arrived, the charging of the capacitor 222 is terminated (step S8), and the output of the first waveform pulse is stopped (step S9). Then, the output of the second waveform pulse is initiated (step S10).

Finally, the output of the second waveform pulse is stopped (step S11), and then the defibrillation pulse generating process is terminated.

As described above, according to the defibrillation system 2 of the present embodiment, the SWs 231 to 236 are controlled by the control unit 230 to output the first waveform pulse having a lower voltage than the second waveform pulse a plurality of times at time intervals, prior to outputting the second waveform pulse, and to superimpose a discharge period of the capacitor 221 (a period for which the first waveform pulse is output) on a charge period of the capacitor 222 (a period for which the power for generating the second waveform pulse is charged).

By outputting the first waveform pulse a plurality of times in this way, it is possible to obtain defibrillation effects even if the voltage of the first waveform pulse is lowered, and to reduce the charge period of the capacitor 221 for outputting the first waveform pulse. Thus, during the charge period of the capacitor 222 for outputting the second waveform pulse, it is possible to output the first waveform pulse, and to early initiate treatment of the heart. Further, by outputting the first waveform pulse prior to outputting the second waveform pulse, it is possible to lower the voltage required for the defibrillation (the voltage of the second waveform pulse), and to effectively perform the defibrillation.

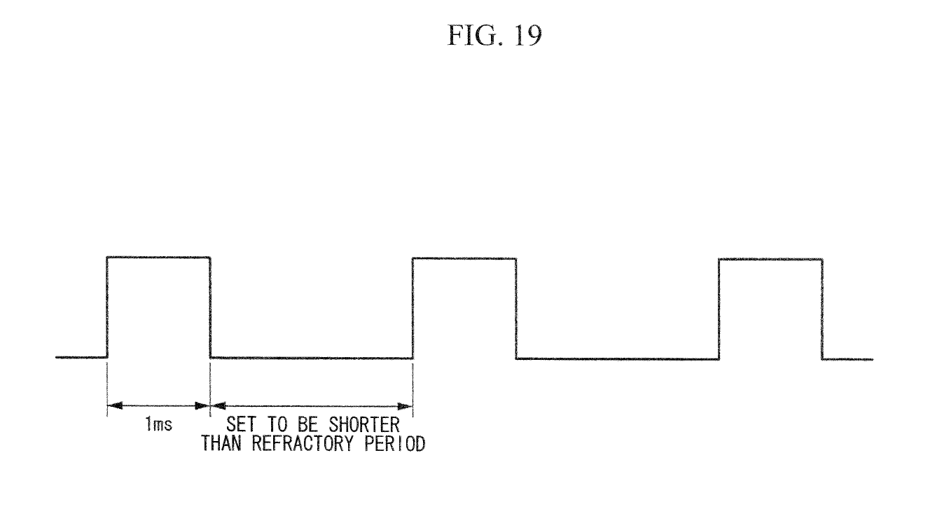

Further, in the defibrillation system 2 of the present embodiment, as shown in FIG. 19, the plurality of first waveform pulses of the low voltage may be continuously output at shorter intervals than a refractory period of the heart. Here, the refractory period of the heart refers to a period for which the heart does not react to any stimulus immediately after the ventricular excitement. Accordingly, by continuously outputting the plurality of first waveform pulses at shorter intervals than the refractory period of the heart, it is possible to obtain effects similar to the case where a normal voltage is applied to the heart. Thus, it is possible to reduce power consumption accompanied by the output of the first waveform pulse.

In detail, as shown in FIG. 19, for example, the first waveform pulse is output for a period of 1 msec to a degree of 10 V to 90 V (an H level), and the output of the first waveform pulse is stopped for a period set to a shorter period than the refractory period of the heart (an L level). Thus, it is possible to obtain effects similar to the case where a normal voltage is applied to the heart, and to reduce power consumption accompanied by the output of the first waveform pulse by an amount of the stop period. Further, the output period of the first waveform pulse is not limited to 1 msec, and it need only be set to be longer than a period for which the heart makes a response.

### First Modification of Third Embodiment

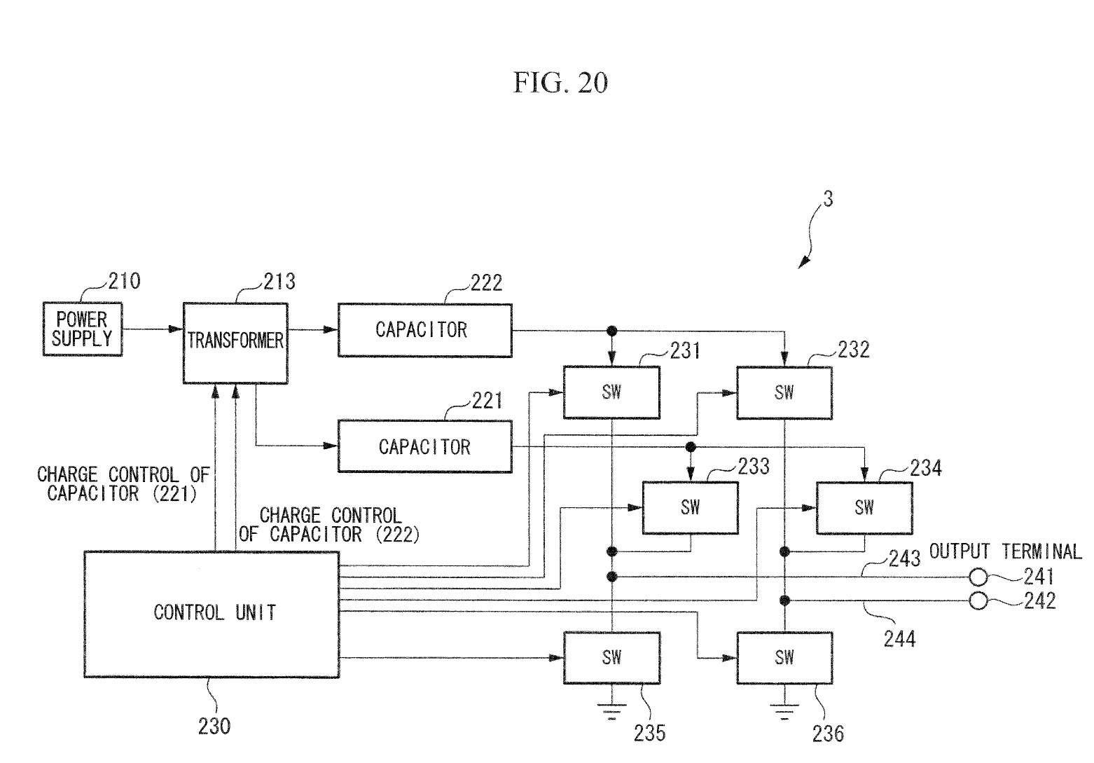

As a first modification of the defibrillation system 2 according to the present embodiment, as shown in FIG. 20, a transformer for applying voltage to capacitors 221 and 222 may be used in common.

The defibrillation system 3 according to the present modification, as shown in FIG. 20, is configured so that the transformer 213 is connected to primary sides of the capacitor 221 for generating a first waveform pulse of a low voltage and the capacitor 222 for generating a second waveform pulse of a high voltage. Voltages are applied to each of the capacitors 221 and 222 by the transformer 213.

Further, a control unit 230 performs the charge control to the capacitor 221 and the charge control to the capacitor 222 on the transformer 213.

According to the defibrillation system 3 according to the present modification, in addition to effects similar to the aforementioned defibrillation system 2, the transformer for the capacitors 221 and 222 can be provided in common, and thus it is possible to miniaturize an apparatus.

### Second Modification of Third Embodiment

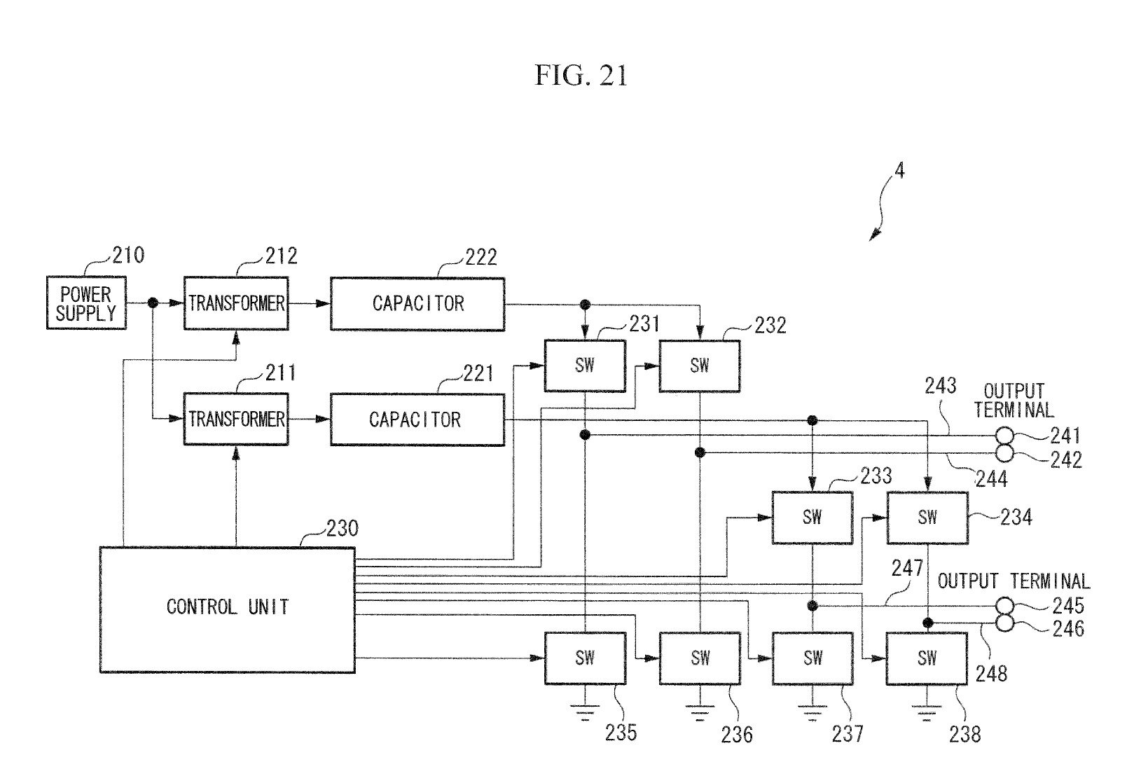

As a second modification of the defibrillation system 2 according to the present embodiment, as shown in FIG. 21, two pairs of output terminals that are connected to the outside (electrodes) may be provided.

The defibrillation system 4 according to the present modification, as shown in FIG. 21, includes, in addition to the configuration similar to the aforementioned defibrillation system 2 (see FIG. 15), output terminals 245 and 246 that are connected to the outside (electrodes), and SWs 237 and 238 that are installed on a circuit between capacitors 221 and 222 and the output terminals 245 and 246.

In the defibrillation system 4 according to the present modification, a lead (not shown) is connected to the output terminals 245 and 246, like the output terminals 241 and 242. Another end of the lead is connected to electrodes (not shown) disposed on the heart. Thus, a pulse generated from the defibrillation system 4 is transferred to the electrodes disposed on the heart. The heart is stimulated by this pulse, and thus fibrillation in the heart is removed.

SWs 231 to 238 are switches that are installed on a circuit between the capacitors 221 and 222 and the output terminals 241 and 242 and a circuit between the capacitors 221 and 222 and the output terminals 245 and 246. The SWs 231 to 238 are turned ON/OFF, thereby switching ON/OFF of each circuit to output pulses stimulating the heart from the output terminals 241 and 242 and the output terminals 245 and 246.

The SWs 231 and 232 are connected to the capacitor 222, and are connected to the SWs 235 and 236, respectively.

The SWs 233 and 234 are connected to the capacitor 221, and are connected to the SWs 237 and 238, respectively.

The SWs 235, 236, 237 and 238 are each connected to the ground.

A wiring 243 is connected between the SWs 231 and 235, and another end of the wiring 243 is connected to the output terminal 241.

A wiring 244 is connected between the SWs 232 and 236, and another end of the wiring 244 is connected to the output terminal 242.

A wiring 247 is connected between the SWs 233 and 237, and another end of the wiring 247 is connected to the output terminal 245.

A wiring 248 is connected between the SWs 234 and 238, and another end of the wiring 248 is connected to the output terminal 246.

A control unit 230 controls ON/OFF operation of the SWs 231 to 238 connected as described above, and controls charging to the capacitors 221 and 222 by means of transformers 211 and 212.

According to the defibrillation system 4 according to the present modification, a second waveform pulse of a high voltage can be output from the output terminals 241 and 242, and a first waveform pulse of a low voltage can be output from the output terminals 245 and 246. In this manner, the output terminals for the first waveform pulse and the output terminals for the second waveform pulse are divided, and the electrodes connected to the output terminals respectively are disposed at different positions. Thereby, it is possible to stimulate different positions using respective pulses.

In detail, for example, the output terminals 241 and 242 are connected to an RV defibrillation electrode, and the output terminals 245 and 246 are connected to a coronary sinus (CS) defibrillation electrode or a superior vena cava (SVC) defibrillation electrode. In this manner, according to the defibrillation system 4 according to the present modification, it is possible to give stimuli to proper positions in vivo by means of the first waveform pulse and the second waveform pulse, and to effectively perform the defibrillation.

### Third Modification of Third Embodiment

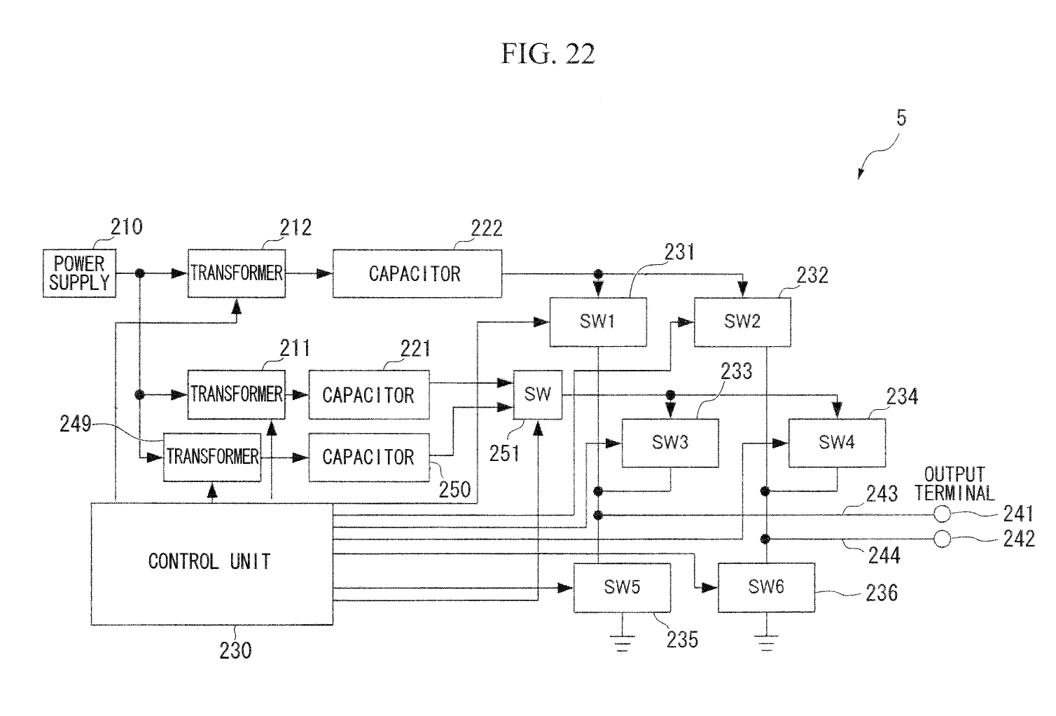

As a third modification of the defibrillation system 2 according to the present embodiment, as shown in FIG. 22, a plurality of capacitors for generating a first waveform pulse may be prepared to switch the capacitors alternately used for an output of the pulse.

The defibrillation system 5 according to the present modification, as shown in FIG. 22, includes, in addition to the configuration similar to the aforementioned defibrillation system 2 (see FIG. 15), a transformer 249 and a capacitor 250 that are connected in parallel to a transformer 211 and a capacitor 221, and an SW 251 that switches the capacitor 221 and the capacitor 250, both of which are capacitors used to generate a first waveform pulse.

In the defibrillation system 5 according to the present modification, electric charges are accumulated on the capacitor 250 via the transformer 249. The SW 251 switches connections of the capacitor 221 and the capacitor 250 to supply voltage to SWs 233 and 234.

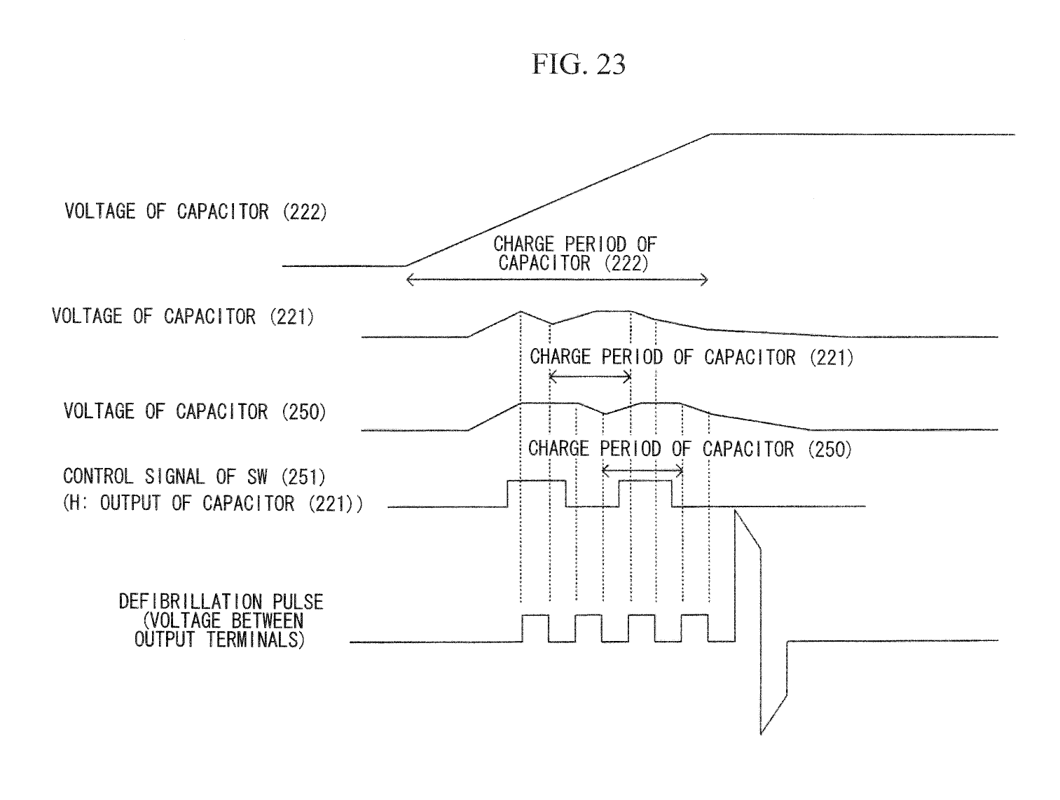

In detail, as shown in FIG. 23, on outputting the first waveform pulse, the capacitor 221 charges voltages of first and third pulses in the plurality of first waveform pulses, and the capacitor 250 charges voltages of second and fourth pulses in the plurality of first waveform pulse.

A control unit 230 controls the SW 251 such that the electric charges charged to the capacitors 221 and 250 are output at different periods. Thus, the SW 251 is configured so that the first and third pulses of the first waveform pulse output the electric charges charged to the capacitor 221 as the first waveform pulse, and the second and fourth pulses of the first waveform pulse output the electric charges charged to the capacitor 250 as the first waveform pulse.

According to the defibrillation system 5 of the present modification which has the aforementioned configuration, since a long charge time can be set for each of the capacitors 221 and 250, it is possible to reliably generate a necessary pulse voltage.

The embodiments of the present invention have been described in detail with reference to the drawings. However, specific configurations are not limited to the embodiments and may include any design in the scope without departing from the subject matter of the present invention. For example, the present invention may be applied to embodiments that properly combine the aforementioned embodiments and modifications.

According to the defibrillation system of the present invention, it is possible to perform the defibrillation more reliably without depending on individual differences of the patient.

Further, according to the defibrillation system of the present invention, it is possible to improve the effects of the defibrillation, and to initiate the treatment of the heart in its early stage.

What is claimed is:

1. A defibrillation system comprising:
an electrode section that is attached to a heart and applies electrical energy to the heart;
a defibrillator that generates the electrical energy on the basis of a predetermined defibrillation wave; and
a lead that electrically connects the electrode section and the defibrillator,
wherein the defibrillation wave includes
a first wave generating first energy,
a second wave generating second energy higher than the first energy of the first wave behind the first wave, and
an application stop period formed between the first wave and the second wave.

2. The defibrillation system according to claim 1, wherein the defibrillation wave is configured so that a first wave section in which the first wave and the application stop period are formed as a set is set ahead of the second wave a plurality of times.

3. The defibrillation system according to claim 2, wherein the first wave has an application time ranging from 30 msec to 200 msec, and the first wave section has a period that ranges from 130 msec to 600 msec and is a sum of the application time of the first wave and the duration time of the application stop period.

4. The defibrillation system according to claim 1, wherein the first wave has a peak voltage ranging from 10 V to 90 V as absolute value, and the second wave has a peak voltage ranging from 70 V to 500 V as absolute value.

5. The defibrillation system according to claim 2, wherein the plurality of first wave sections are configured so that a polarity of at least one first wave is different from those of the other first waves.

6. The defibrillation system according to claim 1, wherein the second wave is formed by synthesizing a square wave and a biphasic wave continuously.

7. The defibrillation system according to claim 6, wherein the wave of the square wave is same as the wave of the first wave.

8. The defibrillation system according to claim 1, wherein the defibrillator comprises:
a pulse generating section for generating pulses stimulating a heart;
a first capacitor for storing electric power for generating the first wave from the pulse generating section;
a second capacitor for storing electric power for generating the second wave having a higher voltage than the first wave from the pulse generating section;
a voltage applying section for applying voltage to the first and second capacitors; and
a controlling section for controlling the pulse generating section, and
the controlling section controls the pulse generating section so that the pulse generating section outputs the plurality of first waves at time intervals prior to outputting the second wave, and so that a discharge period of the first capacitor overlaps with a charge period of the second capacitor,

9. The defibrillation system according to claim 8, wherein the controlling section controls the pulse generating section to output the plurality of first waves at shorter intervals than a refractory period of the heart.

10. The defibrillation system according to claim 8, wherein the voltage applying section for applying voltage to the first and second capacitors is provided in common.

11. The defibrillation system according to claim 8, wherein
the first capacitor is provided in plural numbers, and
the controlling section controls the pulse generating section to make the discharge periods of the plurality of first capacitors different from one another.

12. The defibrillation system according to claim 8, wherein the controlling section controls the voltage applying section to perform charging for at least part of a stop period between the first waves output a plurality of times.

13. A defibrillation system comprising:
a pulse generating section for generating pulses stimulating a heart;
a first capacitor for storing electric power for generating a first wave from the pulse generating section;
a second capacitor for storing electric power for generating a second wave having a higher voltage than the first wave from the pulse generating section;
a voltage applying section for applying voltage to the first and second capacitors; and
a controlling section for controlling the pulse generating section,
wherein the controlling section controls the pulse generating section so that the pulse generating section outputs the plurality of first waves at time intervals prior to outputting the second wave, and so that a discharge period of the first capacitor overlaps with a charge period of the second capacitor.

14. The defibrillation system according to claim 13, wherein the controlling section controls the pulse generating section to output the plurality of first waves at shorter intervals than a refractory period of the heart.

15. The defibrillation system according to claim 13, wherein the voltage applying section for applying voltage to the first and second capacitors is provided in common.

16. The defibrillation system according to claim 13, wherein
the first capacitor is provided in plural numbers, and
the controlling section controls the pulse generating section to make the discharge periods of the plurality of first capacitors different from one another.

17. The defibrillation system according to claim 13, wherein the controlling section controls the voltage applying section to perform charging for at least part of a stop period between the first waves output a plurality of times.

18. The defibrillation system according to claim 13, further comprising:
a first output terminal outputting the first wave; and
a second output terminal outputting the second wave.

19. A cardiac defibrillation method comprising:
a first energy application process of applying first energy to a heart;
a second energy application process of applying a second energy higher than the first energy to the heart; and
an application stop process, set between the first energy application process and the second energy application process, of stopping application of electrical energy for a predetermined time.

20. The cardiac defibrillation method according to claim 19, wherein the first energy application process has an application time ranging from 30 msec to 200 msec, and the sum of the application time and a time of the application stop process ranges from 130 msec to 600 msec.

21. The cardiac defibrillation method according to claim 19, wherein the first energy application process has a peak voltage ranging from 10 V to 90 V as absolute value, and the second energy application process has a peak voltage ranging from 70 V to 500 V as absolute value.
